(19) United States
(12) Patent Application Publication
(10) Pub. No.: US 2021/0292172 A1
(43) Pub. Date: Sep. 23, 2021

(54) FABRICATION OF BLUE-FLUORESCENT AND NON-TOXIC NANODIAMONDS 9NDs) FROM ATMOSPHERIC PARTICULATE MATTERS

(71) Applicant: COUNCIL OF SCIENTIFIC AND INDUSTRIAL RESEARCH, New Delhi (IN)

(72) Inventors: Binoy K. SAIKIA, Jorhat (IN); Nazrul ISLAM, Jorhat (IN); Tonkeswar DAS, Jorhat (IN); Jatin KALITA, Jorhat (IN)

(21) Appl. No.: 17/204,467

(22) Filed: Mar. 17, 2021

(30) Foreign Application Priority Data
Mar. 18, 2020 (IN) 202011011627

(51) Int. Cl. C01B 32/26; C01B 32/28; C09K 11/01; C09K 11/65; B09B 3/00

(52) U.S. Cl. CPC C01B 32/26; C01B 32/28; C09K 11/01; B82Y 20/00; B09B 3/0016; B09B 3/0083; C09K 11/65

(57) ABSTRACT

The present invention relates to a method for fabrication of blue-fluorescent and non-toxic nanodiamonds from atmospheric particulate matters including total solid suspended particulate matter (TSPM) and particulate matter with size less than 10μ (PM₁₀). Mostly, the present invention provides an efficient mitigation process for particulate pollutant by conversion of these pollutants (PM and TSPM) into non-toxic high-value product such as nanodiamond by using the ultrasonic-assisted chemical oxidation method. This method is environmental friendly, simple, and biocompatible for the production of nanodiamonds from such atmospheric particulate matter.

### CROSS-REFERENCES TO RELATED APPLICATIONS

This application claims the priority of Indian Patent Application, Serial No. 202011011627, filed Mar. 18, 2020 pursuant to 35 U.S.C. 119(a)-(d), the content of which is incorporated herein by reference in its entirety as if fully set forth herein.

### FIELD OF INVENTION

The present invention relates to fabrication of blue-fluorescent and non-toxic nanodiamonds from air pollutants. Mostly, the present invention provides an efficient air pollutant mitigation method by conversion of atmospheric particulate matter including total solid suspended particulate matter (TSPM) and particulate matter size less than 10μ, into non-toxic value added products like nanodiamond by using ultrasonic-assisted chemical oxidation method.

### BACKGROUND OF THE INVENTION

Atmospheric particulate pollution has become the major concern of many cities, affecting human health and climate on a global scale. According to the Global Burden of Disease Report, 4.1 million premature deaths worldwide were caused by the exposure of outdoor particulate matter (PM) in 2016. Atmospheric particulate matter (PM) are solid and liquid particles suspended in the atmosphere, which are receiving an increasing rate of attention world-wide due to their impacts on both human health and climate. The chemical compositions of PM includes primarily two constituents, a refractory light-absorbing elements termed as elemental carbon or black carbon (such as PM and major ions) and organic carbon (such as PAHs (Polycyclic aromatic hydrocarbons), and VOCs (Volatile organic compounds)) which is a mixture of oxygenated compounds or hydrocarbons. Carbon compounds are mostly related to atmospheric PMs including dust particulates. Aerosols are mainly size dependent and can be categorized into different categories depending on its size, such as Total Suspended Particulate Matters ((TSPM, <100 μm), coarse (PM₁₀, <10 μm), fine (PM_{2.5}, <2.5 μm), and ultrafine (PM_{0.1}, <0.1 μm) particles. These atmospheric particles are directly emitted into the air (primary aerosol) or formed through gas-to-particle conversion (secondary aerosol) in the atmosphere. Some carbon nanomaterials like fullerene, nanotube and carbon dots are observed to be present in atmospheric PMs because of the carbon content abundantly present in it. But, the fabrication of nanodiamonds from atmospheric PMs has not been established or studied. Thus, utilization of carbon content from particulate pollutants to produce a value added carbon nanomaterials like nanodiamond product is a better option for mitigating particulate pollution.

In recent years, nanodiamonds (NDs) have been gaining worldwide attention due to their different applications in bio-sensing, biomedical imaging and drug delivery. They are also used in novel wear resistant polymer, lubricant additives, and metal coating due to the presence of some properties like high chemical resistivity, abrasive nature, and super hardness respectively. Nanodiamond is an important member of nano-carbon family with the size ranging from 1 to 100 nm which is very small in size. The family of nanocarbons includes nanodiamonds, graphite, nanotubes and fullerenes. NDs have optical properties, better conductivity of heat, higher electrical resistance and also transparent to visible light, Infra-red, ultraviolet radiation, and X-ray spectrum. Biocompatibility, chemical inertness, fluorescence stability, and biosafety are the main characteristic of ND's. The first artificial NDs were synthesized using detonating carbon-containing explosives methods in absence of oxygen environment. Subsequently, a lots of methods have been discovered to produce nanodiamond such as laser ablation, CVD (chemical vapor deposition), HPHT (high energy ball milling of high-pressure high-temperature) diamond microcrystals, RDX (research department explosive), microplasma-assisted technique of nanodiamond formation using ethanol vapor at atmospheric pressure, chlorination of carbides, ion irradiation of graphite and ultrasound cavitation. Detonation nanodiamond (DND) or ultradispersed diamond (UDD) having the particle size 4-5 nm was synthesized by the detonation of carbon particle explosives. These methods have some limitations for production of NDs from PMs due to the presence various toxic elemental and organic carbon compounds in PMs. Further, the above mentioned methods are time consuming, expensive and it is difficult to segregate the size of the carbon particles in bulk quantities. There is no literature nor studies have been observed for fabrication of nanodiamonds from atmospheric PM (NDs derived from PM₁₀ and TSP).

However, some of the related prior art references in the literature disclose production of some other value added carbon nanomaterials like fullerene, carbon nanotubes and carbon dots from atmospheric PM.

Environ. Sci. Technol. 2012, 46, 1335-1343 (Occurrence of Aerosol-Bound Fullerenes in the Mediterranean Sea Atmosphere) report the presence of fullerene in atmospheric particulate matters for the first time. The atmospheric particles were collected using High Volume Sampler on a Quartz Microfiber Filter papers. They used toluene as a solvent for the extraction of carbon particles and sonicated repeatedly two times for 15 min in ultrasonic water bath. The authors confirmed and quantified the extracted fullerene products based on a new method such as liquid chromatography coupled to mass spectrometry (LCMS) presenting sensitivities between 5.4 and 20.9 pg/m3.

Atmospheric Sci. Let. 2006, 7, 93-95 (Carbon nanotubes in wood soot) discloses that multiwall carbon nanotubes were observed in a small fraction of soot particles which are generated during burning of pine wood in air. The soot particles were collected with the help of High Volume Sampler and thermophoretic precipitator. The authors used Transmission Electron Microscopy technique for characterization and demonstrated that the carbon nanotubes aggregated with other fullerene-like carbon nanoforms and graphite fragments.

Chemosphere. 2016, 164, 84-91 (Nanominerals, fullerene aggregates, and hazardous elements in coal and coal combustion-generated aerosols: An environmental and toxicological assessment) reports that fullerene like materials were observed in both coal and coal combustion generated aerosol (PM₁₀ and PM_{2.5}). Both the size of the particles were collected using air sampler such as Respirable Dust Sampler for PM₁₀ and Fine Particulate Sampler for PM_{2.5}. The authors demonstrated the formation of fullerene like carbon particle in both PM₁₀ and PM_{2.5} with the help of Transmission Electron Microscopy technique and showed curved crystalline disordered graphitic layers which are similar to fullerene.

Chemosphere. 2020, 244, 125519 (An environmental evaluation of carbonaceous aerosols in PM₁₀ at micro- and nano scale levels reveals the formation of carbon nanodots) discloses that carbon nanodots formation was observed in PM₁₀. The authors mentioned that the carbon nanodots were derived from PM₁₀ by using chemical oxidation methods where H₂O₂ was used as an oxidizing agent. The authors also reported that the obtained carbon particles were mainly less than 10 nm with few other elements like Fe and Ca.

CN103482623 provides a direct current arc method for the production of Nanodiamond. It is characterized in that using direct current arc hydrogen plasma as thermal source, graphite is carbon raw material, nickel is catalyzer, silicon is as a method for forming core material synthesized nanometer diamond particle. In this method, the derived nanodiamond products were purified with the process of acid treatment, high temperature oxidation, rinsing, to remove the residual impurities such as metal, graphite, amorphous carbon, silicon carbide, to obtain high-purity diamond nanoparticle.

JP2017001916A provides a method for producing nano-diamond powder with high re-dispersibility. The method reported two steps including a sodium ion addition step and a spray drying step. The nano-diamond powder was obtained by subjecting the nano-diamond-dispersed liquid including the sodium ion added nano-diamond to spray drying. The content of the sodium ions is 0.1 to 1.5 mass percentages.

CN109385275A discloses a method for preparing fluorescent carbon quantum dots by utilizing an organic matter anaerobic fermentation intermediate product as a carbon-based material. The products were obtained by utilizing the organic matter anaerobic fermentation intermediate product as a carbon-based material and performing hydrothermal reaction after a liquid nitrogen source with addition of ultra-pure water.

CN104609393A discloses a simple preparation method of fluorescent carbon quantum dots. The process comprises the following steps: acquiring carbon ash, namely igniting and organic matter precursor and obtaining carbon ash which is generated due to insufficient combustion; dispersing by using an polarity organic solvent, namely mixing the carbon ash obtained in the above step with the polarity organic solvent so that the carbon ash is dispersed in the polarity organic solvent. The next step contains standing and separating to obtain a fluorescent carbon quantum dot colloid dispersed by a polar organic solvent. The organic precursor refers to at least one of 1-octene or ethanol.

CN104071769 provides a method of preparing a fluorescent carbon point by virtue of a chemical oxidation method, the fluorescent carbon point and application of the fluorescent carbon point. The method comprises the steps of (1) oxidation etching step: adding a carbon source material in an oxidizing solution, and carrying out condensation reflux treatment; (2) dialysis desalting step: after the reaction in the step one is finished, adding an alkaline reagent for neutralizing to be faintly acid, and carrying out dialysis desalting to obtain coal-based fluorescent carbon point hydrosol; and (3) drying step: carrying out vacuum drying on the substance obtained in the step two to obtain the fluorescent carbon point in a solid state, wherein the carbon source material is coal or a coke material generated after coal is heated and carbonized.

WO2009038850A2 provides a method for fabrication of functionalized nanodiamonds. The document also provides composites including nanodiamonds and polymers; and electrospun fibers including nanodiamonds and polymers. In this document, organic solvents like toluene, dichloromethane, benzene, dichloromethane, n-n dimethylformamide, acetone, and ethanol are used for fabrication of nanodiamonds which are not ecofriendly.

WO2014174150A1 relates to a method for producing zeta negative single digit carboxylated nanodiamond dispersion. The method comprises adjusting pH of zeta negative carboxylated nanodiamond suspension to at least 7, and zeta negative single digit carboxylated nanodiamond dispersion is over −37 mV measured at pH over 7. However, the NDs suspensions are stable with Zeta potentials in the range of −24 mV to −25 mV.

EP2535312A2 relates to a nano-diamond dispersion solution and a method of preparing the same. The method comprised: providing a nano-diamond aggregation; mixing the nano-diamond aggregation with a metal hydroxide solution and stirring the mixture such that the nano-diamond aggregation was separated, to obtain a mixture solution; stabilizing the mixture solution such that the mixture solution was separated into a supernatant and precipitates; and extracting the supernatant and precipitates.

WO2013160704A1 provides a method for the separation of diamond particle clusters into discrete diamond particles and/or into smaller diamond particle clusters comprising fewer diamond particles. The diamond particle clusters combined with at least one liquid phase organic or inorganic compound, or with a solution of at least one organic or inorganic compound in at least one solvent to form a reaction mixture. It means they used to separate the diamond particle clusters into discrete diamond particles and/or into smaller clusters within the reaction mixture to produce diamond particles with dangling bonds or free bonding sites on the surface of the diamond particles.

CN108862273B provides a preparation method of a nano-diamond colloid and a secondary dispersion method of nano-diamond. The process comprises the following steps: acidizing the nano-diamond raw material, dispersing the acidized nano-diamond raw material in n-octane, and mechanically grinding the acidized nano-diamond raw material to obtain a clear and transparent black colloidal solution; drying the obtained colloidal solution to obtain a paste with the mass percent of the nano-diamond of 60-70%; the paste is dissolved in n-octane to obtain clear and transparent black colloidal solution again, so that secondary dispersion of the nano-diamond was realized. The nano-diamond colloidal solution prepared by the method can be applied to the fields of precision grinding, polishing, composite materials, lubricating oil. Journal of Photochemistry & Photobiology, B: Biology. 2019, 195, 1-11 (Blue-fluorescent and biocompatible carbon dots derived from abundant low quality coals) reports an oxidative chemical method for synthesis of high-value carbon dots (CDs) from cheap and abundant low-quality high-sulfur coals for use in high-end applications. These CDs were synthesized by using wet-chemical ultrasonic stimulation induced process which is environmentally facile and less drastic compared to other chemical methods of production of CDs. The sizes of the synthesized CDs from different types of coal samples were estimated to be in the range of 1-4 nm, 1-6 nm, 2-5 nm, and 10-30 nm. The quantum yield (QY) of the CDs was determined and it was found to be around 3-14%. For high-end field application, the CDs were further tested for toxicity and were reported to be safe for environmental and biological applications.

In the light of the above existing prior arts, the formation of carbon nanomaterials like fullerene, carbon nanotube and carbon nanodots from atmospheric particulate matters are established using different characterization techniques. However, there is no systematic procedure observed for the synthesis of those carbon nanomaterials easily and also no studies have been observed for production of non-toxic nanodiamonds from air pollutants (PMs). In above m cited literature, different types of methods are illustrated for the production of nanodiamonds from different carbonaceous sources. Some references use hydrothermal process to synthesize carbon nanomaterials which is time consuming. A preparation of nontoxic nanodiamonds from atmospheric particulate matter is very challenging due to the presence of heterogeneous mixture of atmospheric carbonaceous contaminants. Therefore, there is a need in the art for the process for preparing non-toxic nanodiamonds, which is simple, easy to control and environment friendly.

### Objectives of the Invention

The main objective of the present invention is to fabricate blue-fluorescent and non-toxic nano-diamonds from atmospheric particulate matter (TSPM and PM₁₀).

Another objective of the present invention is to provide an air pollutant mitigation method for conversion of air pollutants consisting of atmospheric particulate matter into non-toxic Nanodiamond (NDs) for biological applications.

### SUMMARY OF THE INVENTION

As aspect of present invention provides a method for preparing nanodiamonds from atmospheric pollutants comprising the steps of:
a) collecting atmospheric pollutants;
b) mixing the atmospheric pollutants obtained in step (a) with hydrogen peroxide to obtain an oxidized particulate matter;
c) ultrasonicating the oxidized particulate matter obtained in step (b) to obtain a first mixture;
d) filtering the first mixture obtained in step (c) using a polytetrafluoroethylene membrane filter (0.22 μm) to obtain a filtrate;
e) centrifuging the filtrate obtained in step (d) to obtain a supernant;
f) treating the supernatant obtained in step (e) with HNO₃/H₂SO₄ acid and heating to obtain an extract;
g) neutralizing the extract obtained in step (f) by adding ammonia solution drop wise to obtain a second mixture;
h) filtering the second mixture obtained in step (g) using an ultrafiltration (1 KDa) to obtain a filtrate solution; and
i) concentrating the filtrate solution obtained in step (h) to obtain the nanodiamonds.

Another aspect of present invention provides a nanodiamond prepared by the method of the present invention.

In an aspect, the present invention provides a nanodiamond having a diameter in the range of 3-24 nm and a X-ray diffraction pattern showing peaks at the d-spacings listed in Table A:

TABLE A

| ‘d’ spacing value (Å) | 2θ (°) |
|---|---|
| 2.0 | 43.22 |
| 1.26 | 75.1 |

In another aspect, the present invention provides a nanodiamond having oxygen-containing hydrophilic functional groups and blue-fluorescence under UV-light, wherein said nanodiamond are non-toxic and bio-compatible.

Yet another aspect of the present invention provides a nanodiamond for use in bio-sensing, biomedical imaging, drug delivery, wear resistant polymer, lubricant additives, and metal coating.

### BRIEF DESCRIPTION OF THE DRAWINGS

FIG. 1: Schematic diagram of Environmental sampler used for the collection of atmospheric particulate matter. FIG. 1a shows the High volume sampler containing 1. Flow meter, 2. Valve, 3. timer, 4. Pump, 5. Outlet, 6. Filter paper containing TSPM. FIG. 1b shows the Respirable Dust Sampler containing 1. Air Inlet, 2. Flow meter, 3. Cyclone assembly, 4. Dust sample deposited chamber, 5. Suction device 6. Timer, 7. Pump, 8. Filters containing PM₁₀.

FIG. 2 represent the process for the preparation of Nanodiamonds (NDs) from atmospheric pollutants (PM₁₀ and TSPM) in accordance with the invention.

FIG. 3 Flow sheet provides a technique of nanodiamond fabrication in large scale quantity for commercial use. The steps comprise: 1. Collection of air pollutants using Air sampler. 2. Mixing tank with ultrasonicator. 3. Filtration unit. 4. Centrifuge. 5. Mixing tank. 6. Ultra-filtration. 7. Rotary Evaporator.

FIG. 4 Provides the Transmission Electron Micrograph of nanodiamond (NDs) prepared from TSPM according to the present invention. FIG. 4a shows the nanodiamonds prepared from TSPM. FIG. 4b shows the elements (C and O) present in fabricated product. FIG. 4c shows Fast Fourier Transform (FFT) Pattern and, FIG. 4d shows the size distribution of NDs derived from TSPM.

FIG. 5 Provides the High resolution Transmission Electron Micrograph of the NDs prepared from TSPM according to the present invention. FIGS. 5a and b show the HRTEM images of nanocrystal as highlighted by hexagon. FIGS. 5c and d shows FFT pattern showing the hexagonal crystalline structure of the carbon particle. FIGS. 5e and f show the Elemental mapping images indicate presence of C and O element.

FIG. 6 Provides data relating to characterizations techniques like XRD (FIG. 6a), FTIR (FIG. 6b), and Raman (FIG. 6c) of the NDs prepared from TSPM.

FIG. 7 Provides the data relating to optical properties of NDs prepared from TSPM. FIG. 7a shows UV-visible spectra of NDs prepared from TSPM. FIG. 7b shows FL spectra of NDs prepared from TSPM. FIG. 7c-i shows the NDs in day light and 7c-ii shows the ND sample under UV light at 365 nm.

FIG. 8 Provides the Transmission Electron Micrograph of the NDs prepared from PM₁₀ according to the present invention. FIG. 8a shows the nanodiamonds prepared from PM₁₀. FIG. 8b shows the elements (C and O) present in fabricated product. FIG. 8c shows Fast Fourier Transform (FFT) Pattern and, FIG. 8d shows the size distribution of ND obtained from PM₁₀.

FIG. 9 Provides the High resolution Transmission Electron Micrograph of the ND prepared from PM₁₀ according to the present invention. FIGS. 9a and b shows the HRTEM images of carbon nanocrystal. FIGS. 9c and d shows FFT pattern showing the hexagonal crystalline structure of the carbon particle.

FIG. 10 provides data relating to the other characterizations techniques like XRD (FIG. 10a), FTIR (FIG. 10b), and Raman (FIG. 10c) of the NDs prepared from PM₁₀.

FIG. 11 Provides the data relating to optical properties of NDs prepared from PM₁₀. FIG. 11a shows UV-visible spectra of ND prepared from PM₁₀. FIG. 11b shows FL spectra of ND prepared from PM₁₀. FIG. 11c-i shows the ND in day light and 11c-ii shows the ND under UV light at 365 nm.

FIGS. 12A-12B provide the zeta potential analysis data of NDs prepared from TSPM (Figure a) and PM₁₀ (Figure b) for the determination of surface properties.

FIGS. 13A-13B provide the data relating to toxicity of NDs at cellular and genetic level derived from both TSPM (NDs coded as S1) and PM₁₀ (NDs coded as S2).

### DETAILED DESCRIPTION OF THE INVENTION

The present invention relates to a method for preparation of blue-fluorescent and nontoxic nanodiamonds from atmospheric pollutants (SPM and PM₁₀) with the help of ultrasonic-assisted chemical oxidation method.

The present invention is related to use of air pollutants as a source of carbon and conversion of same into value added carbon nanomaterials such as nanodiamonds (NDs). The carbon source is mostly vehicular exhaust diesel particulate matter. The method comprises chemical oxidation with the help of Hydrogen peroxide (30%) which is considered as environment friendly. The formed nanodiamond products are functionalized with a hydroxyl groups, carbonyl group, carboxyl group etc. Therefore, it shows fluorescence properties and the product is non-toxic and biocompatible.

The present invention provides a method for preparing nanodiamonds from atmospheric pollutants comprising the steps of:
a) collecting atmospheric pollutants;
b) mixing the atmospheric pollutants obtained in step (a) with hydrogen peroxide to obtain an oxidized particulate matter;
c) ultrasonicating the oxidized particulate matter obtained in step (b) to obtain a first mixture;
d) filtering the first mixture obtained in step (c) using a polytetrafluoroethylene membrane filter (0.22 μm) to obtain a filtrate;
e) centrifuging the filtrate obtained in step (d) to obtain a supernant;
f) treating the supernatant obtained in step (e) with HNO₃/H₂SO₄ acid and heating to obtain an extract;
g) neutralizing the extract obtained in step (f) by adding ammonia solution drop wise to obtain a second mixture;
h) filtering the second mixture obtained in step (g) using an ultrafiltration (1 KDa) to obtain a filtrate solution; and
i) concentrating the filtrate solution obtained in step (h) to obtain the nanodiamonds.

In an embodiment of the present invention, there is provided a method for preparing nanodiamonds from atmospheric pollutants, wherein the atmospheric pollutants comprise Total Suspended Particulate Matter having the particle size less than 100 μm (TSPM) and Particulate Matter having the size of aerodynamic diameter of 2.5-10 μm (PM₁₀).

In another embodiment of the present invention, there is provided a method for preparing nanodiamonds from atmospheric pollutants, wherein the atmospheric pollutants are collected using High Volume Sampler or Respirable Dust Sampler on quartz filter papers.

In yet another embodiment of the present invention, there is provided a method for preparing nanodiamonds from atmospheric pollutants, wherein the hydrogen peroxide used in step (b) is 30%.

In still another embodiment of the present invention, there is provided a method for preparing nanodiamonds from atmospheric pollutants, wherein the ultrasonication in step (c) is carried out for 1 hour at room temperature.

In an embodiment of the present invention, there is provided a method for preparing nanodiamonds from atmospheric pollutants, wherein the centrifugation in step (e) is carried out at 1400 rpm for 1 hour.

In another embodiment of the present invention, there is provided a method for preparing nanodiamonds from atmospheric pollutants, wherein heating in step (f) is carried out at 55-60° C. for 30 minutes.

An embodiment of the present invention provides nanodiamond prepared by the method of the present invention.

In another embodiment of the present invention, there is provided nanodiamond having a diameter in the range of 3-24 nm and a X-ray diffraction pattern showing peaks at the d-spacings listed in Table A:

TABLE A

| ‘d’ spacing value (Å) | 2θ (°) |
|---|---|
| 2.0 | 43.22 |
| 1.26 | 75.1 |

In yet another embodiment of the present invention, there is provided nanodiamond having a diameter in the range of 3-6 nm and a X-ray diffraction pattern showing peaks at the d-spacings listed in Table A:

TABLE A

| ‘d’ spacing value (Å) | 2θ (°) |
|---|---|
| 2.0 | 43.22 |
| 1.26 | 75.1 |

In still another embodiment of the present invention, there is provided nanodiamond having a diameter in the range of 10-24 nm and a X-ray diffraction pattern showing peaks at the d-spacings listed in Table A:

TABLE A

| ‘d’ spacing value (Å) | 2θ (°) |
|---|---|
| 2.0 | 43.22 |
| 1.26 | 75.1 |

In another embodiment of the present invention, there is provided nanodiamond having oxygen-containing hydrophilic functional groups and blue-fluorescence under UV-light.

In yet another embodiment of the present invention, there is provided nanodiamond, wherein said nanodiamond are non-toxic and bio-compatible.

Another embodiment of the present invention provides a nanodiamond for use in bio-sensing, biomedical imaging, drug delivery, wear resistant polymer, lubricant additives, and metal coating.

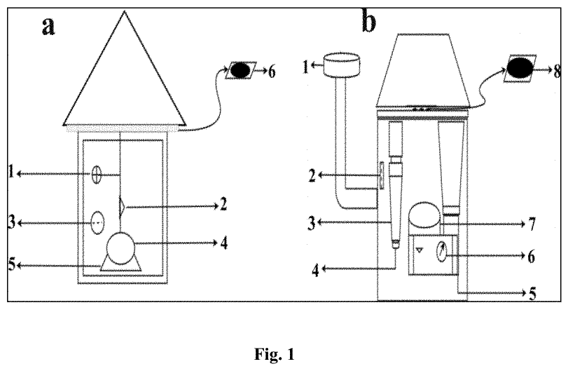

The present invention provides a method of producing nanodiamonds from a carbon source, such as atmospheric air pollutant (TSPM and PM₁₀). These air pollutants are collected by using environmental samplers like Respirable Dust Sampler for PM₁₀ and High volume sampler for Total Suspended Particulate Matter (TSPM) which are illustrated in FIG. 1.

The method of the present application is a feasible technique to remove associated atmospheric contaminants from particulate matter and convert them into nanodiamonds using chemical oxidation (H₂O₂ as an oxidizing agent) followed by ultrasonication process. This method requires less time and helps to separate carbon particles from impurities of Particulate Matters. The derived nanodiamond are confirmed by using different characterization techniques such as High resolution-transmission electron microscopy (HR-TEM; JEOL JEM 2100), X-ray diffraction (XRD; Rigako, Ultima IV), Raman spectroscopy (Horiba Jobin Vyon, Model LabRam HR), Fourier transforms infrared spectroscopy (FT-IR; System-2000, Perkin-Elmer), X-ray Photoelectron Spectrometer (XPS; ESCALAB Xi+), ultraviolet-visible spectroscopy (UV-visible; Analytik Jena, SPECORD200, Germany), fluorescence spectroscopy (FL; Horiba Fluorlolog-3), and Zeta potential (ZETASIZER; Model-Nano ZS, Malvern, UK).

### EXAMPLES

### Example 1: Fabrication Process of Nanodiamonds (NDs) from Total Suspended Particulate Matter (TSPM) and PM₁₀

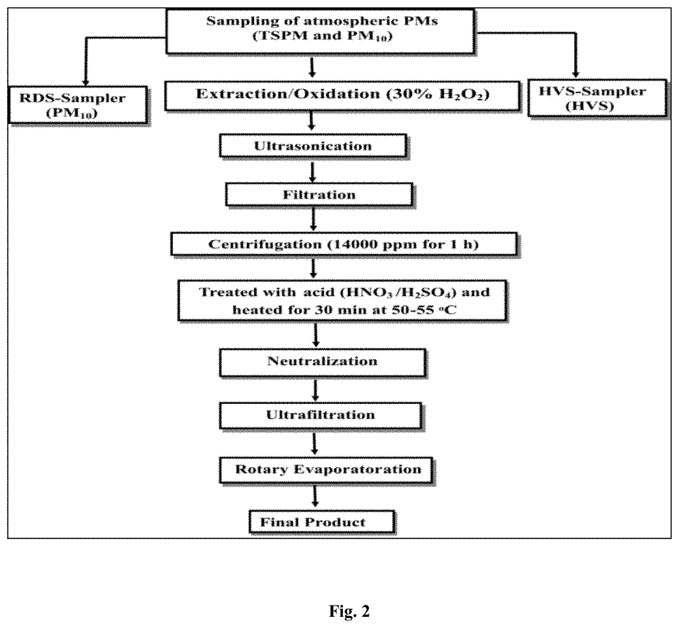

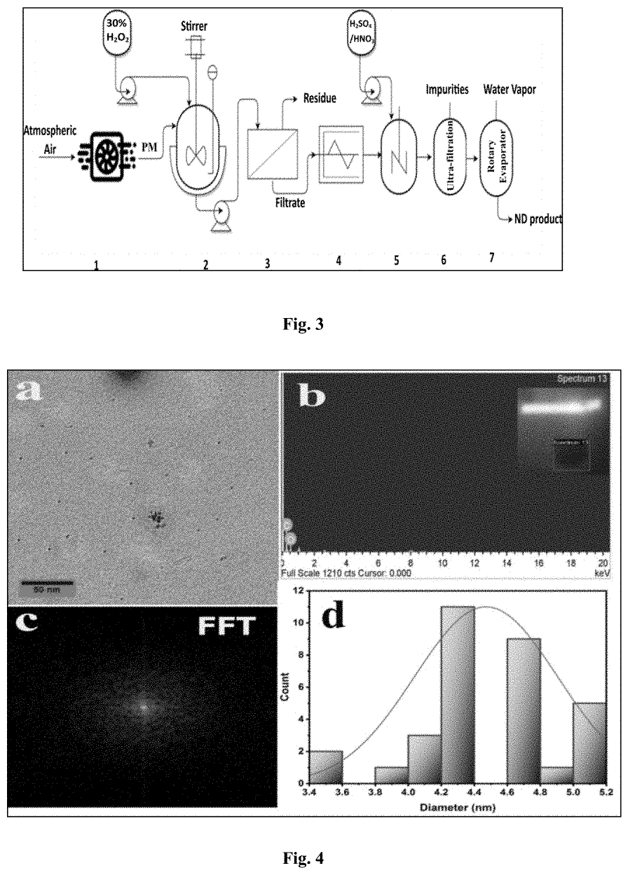

The ambient air containing particulate pollutants with the size ranges from 10-100 μm in diameter (TSPM) are used for fabrication of blue fluorescent nanodiamonds. The collected TSPM (5-6 g) was mixed with 100 mL of hydrogen peroxide (20-30%) in a Teflon beaker and the mixture was then ultrasonicated (frequency: 20 kHz) in a microprocessor-based ultrasonicator (Model-Power Sonic) for about 1 hrs at an atmospheric pressure and temperature. Polytetrafluoroethylene membrane filter (˜0.22 μm) was used to filter the resultant mixture. The filterate was then centrifuged at about 1400 rpm for 1 hour. The supernatant was carefully taken and treated with nitric acid to remove the atmospheric contaminants or impurities. The nitric acid treated extract was then neutralized by adding ammonia solution drop wise. This neutral extract was concentrated using ultrafiltration and rotary evaporation and kept in a refrigerator at 4° C. for subsequent analysis (FIGS. 2 and 3).

FIG. 4 illustrates the micrographs of the Transmission Electron Microscopic Technique used to determine the microstructure/nanostructure of nanodiamonds prepared from TSPM. The micrographs clearly show the formation of nanodiamonds. The formed nanodiamonds are unagglomerated types (FIG. 4a) and distributed uniformly sized with a crystalline phase which is confirmed by Fast Fourier Transform (FFT) pattern [Fourier transforms infrared spectroscopy (FT-IR; System-2000, Perkin-Elmer)] (FIG. 4c). The chemical composition was also determined by using energy-dispersive spectroscopy which indicated that the TSPM derived NDs predominantly comprises of carbon and oxygen (FIG. 4b) and also free from impurities. The diameter of the nanodiamonds was estimated to be in the range of 3-6 nm (FIG. 4d).

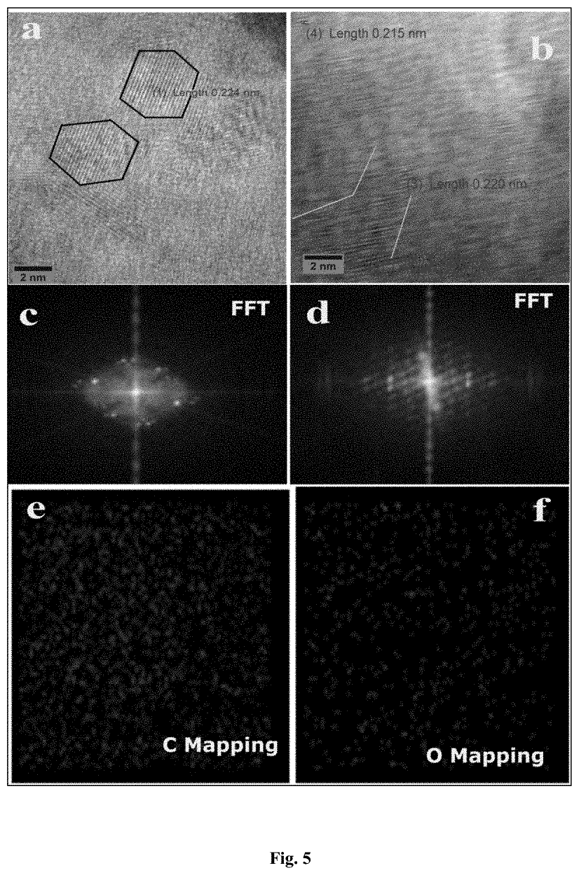

At high-resolutions, electron beam analysis (HRTEM) [JEOL JEM 2100] of nanodiamond prepared from TSPM is illustrated in FIG. 5. The interplanar spacing (d spacing) of the crystal lattice was found to be in the range of 0.220-0.266 nm (2.20-2.66 Å) in the micrograph (FIGS. 5a and 5b) which indicates the presence of nanodiamond. These results are found to be in good agreement with the diamond phases of a cubic structure having lattice planes (111). The FFT image also revealed that the particles are hexagonal with a crystalline structure (FIGS. 5c and 5d). The elemental mapping of carbon and oxygen are also shown in FIGS. 5e and 5f, respectively. The TEM/HRTEM images, elemental characterizations, and FFT measurements of the interplaner lattice plane revealed the presence of nanodiamond particles in the TSPM derived samples rather than other carbon particles.

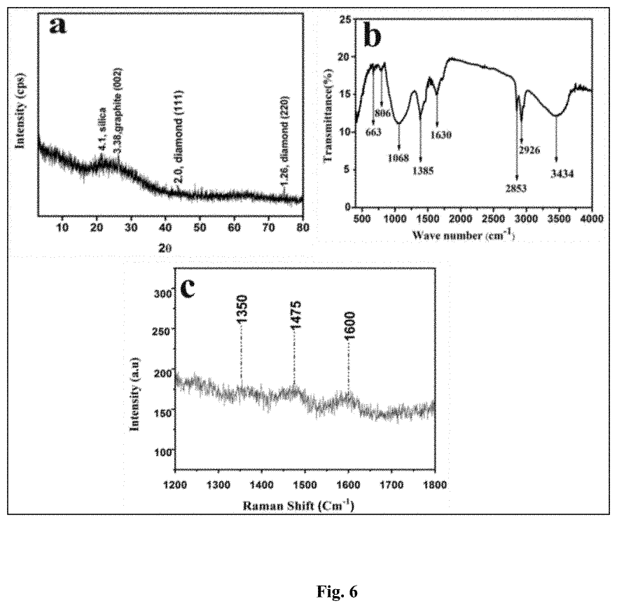

The X-ray diffraction (XRD) [Rigako, Ultima IV] analysis of a nanodiamond prepared from TSPM are shown in FIG. 6a, confirming the crystallites of the nanodiamond having a plane of the cubic structure. A broad peak observed at the ‘d’ spacing value of 4.16 Å) (2θ=21.3° indicates the presence of silica substrate and the second peak found at 2θ=26.3° (d spacing value=3.38 Å) belongs to the crystal plane of graphite (002). The peak at 20°-30° corresponded to the plane (002) of crystal graphite. The broad peak at 26° attributed to the nanodiamonds surrounded by an amorphous carbon matrix with abundant oxygen-containing functional. The other peaks observed at 2θ=43.22° (d spacing value=2.0 Å) and 2θ=75.1° (d spacing value=1.26 Å) corresponding to the (111) and (220) cubic planes of the diamond, which indicated that the structure of TSPM-derived NDs crystal is cubic. This analysis revealed that the formed NDs consist of both sp² and sp³ hybridized carbon structure.

FIG. 6b illustrates the FT-IR (System-2000, Perkin-Elmer) spectral analysis of the TSPM-derived NDs. The peak observed at 660-685 cm⁻¹ is due to the absorption of C—H bonds at the sp³ hybridized carbon atom. The peak observed at 806 cm⁻¹ is assigned to the NO₂ group. The absorption peak observed in the range of 1027-1068 cm⁻¹ and 1385-1400 cm⁻¹ is due to the stretching vibration of C—O and OH group along with other oxygen-containing functional groups, respectively. The absorption band is found within the range of 1630 cm⁻¹ corresponding to the bending vibration of hydroxyl (O—H) and carbonyl (C═O). In this investigation, the absorption bands observed for the hydroxyl groups are found to be more predominant over the carbonyl groups which indicate high solubility in water.

The Raman spectra (Horiba Jobin Vyon, Model LabRam HR) show mainly three absorptions bands for TSPM-derived NDs samples (FIG. 6c). The NDs prepared from TSPM samples is evident from the peak at 1320 cm⁻¹, which is due to the phonon confinement effect. The first Raman peak ranging from 1320-1350 cm⁻¹ indicating near the position of D-band for sp³ hybridized carbon. The obtained diamond particle exhibits mainly Raman scattering peak at 1333 cm⁻¹. The second peak in the Raman spectra occurs in the range of 1435-1475 cm⁻¹ specifies the D-band corresponding to the sp³ hybridised structure of carbon. The third peak observed at 1600 cm⁻¹ and 1620 cm⁻¹ referred to as G-band attributed to the sp² structure of carbon. The Raman analysis revealed that the nanodiamond particles are dominantly present in the TSPM-derived sample rather than that of graphite.

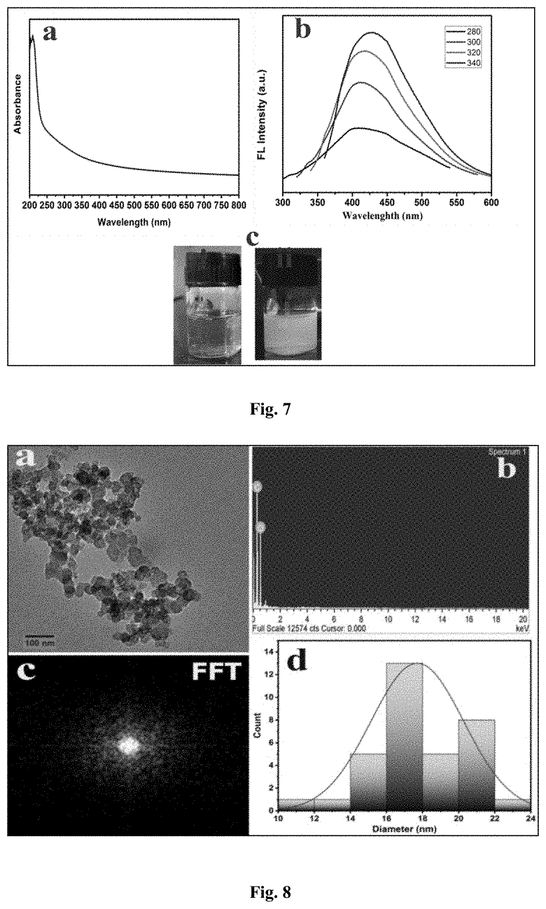

The photo-optical properties of the TSPM-derived ND sample were investigated by using ultraviolet (UV-Vis) spectroscopy and FL spectroscopy (UV-visible; Analytik Jena, SPECORD200, Germany), fluorescence spectroscopy (FL; Horiba Fluorlolog-3). FIG. 7a shows the UV-visible spectroscopic analysis of the NDs samples obtained from TSPM samples. The absorption peak observed at 210 nm (5.9 eV) is due to the π-π* and n-π* transition of C═C and C═O bonds present in the NDs samples, respectively. This peak attributed to the intrinsic absorption of nanodiamond that is larger than a diamond of 5.5 eV because of minor size-induced blue shift.

FIG. 7b shows the FL spectra of produced NDs samples under the excitation wavelength of 280-340 nm, at an increment of 20 nm. The FL properties of the TSPM derived NDs sample is observed to be excitation dependent as depicted in FIG. 7b. With the increase of excitation wavelength, the FL spectra of NDs sample is observed to be shifted from red to green and yellow regions which signify as a major characteristic of nanodiamond. This phenomenon is occurred due to the presence of numerous fluorophore or chromophore systems (aromatic and oxidation groups) in NDs, which is also confirmed from the FT-IR as discussed above.

The TSPM-derived NDs samples are found to be blue-fluorescence under UV-light (at 365 nm) with considerable intensity as shown in FIG. 7c-ii, which is one of the fascinating properties of nanodiamonds.

### Example 2: Fabrication of Nanodiamonds (NDs) from Particulate Matter (PM₁₀)

The same experiment as outline in Example 1 was conducted with the atmospheric particulate matter (PM₁₀ having the aerodynamic sizes of 2.5-10 μm) in the same manner.

FIG. 8, shows the TEM image of nanodiamonds prepared from PM₁₀ which are agglomerated types. TEM images show the formation of different sizes of carbon nanocrystals (FIG. 8a). Energy-dispersive spectroscopy (EDS) clearly shows the presence of carbon and oxygen predominantly in PM₁₀ derived ND samples (FIG. 8b) and also free from impurities with FFT pattern shown in FIG. 8c. The diameter of these nanodiamonds was estimated to be in the range of 10-24 nm (FIG. 8d).

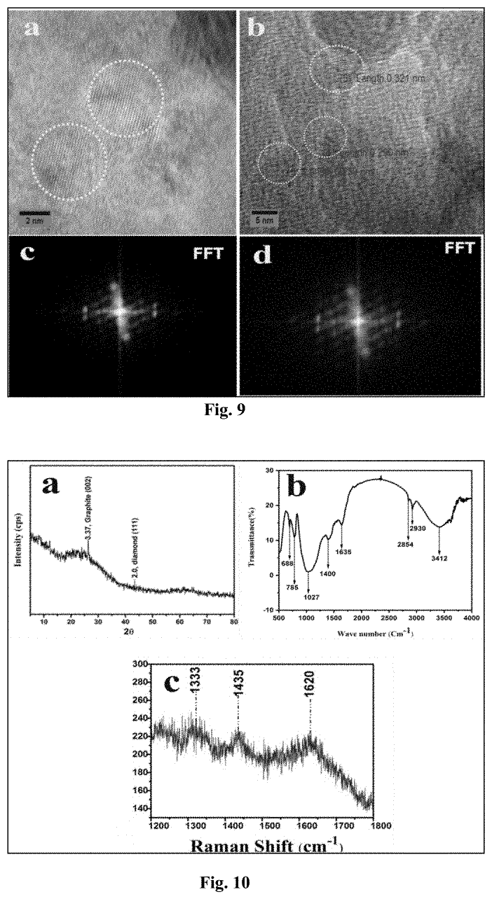

FIG. 9 illustrate the micrograph of HRTEM analysis. The micrograph shows the interplane spacing of 0.218-0.321 nm (2.18-3.21 Å) (FIGS. 9a and b), which revealed the presence of nanodiamond phases. The FFT (FIGS. 9c and d), measurements of the interplaner lattice plane revealed the presence of nanodiamond particles in the PM₁₀-derived samples rather than other carbon particles.

The XRD analysis of PM₁₀ derived ND sample (FIG. 10a) shows a broad peak peak at 26° attributed to the nanodiamonds surrounded by an amorphous carbon matrix with abundant oxygen-containing functional groups. The peak at 20°-30° corresponded to the plane (002) of crystal graphite as the similar results of TSPM derived NDs sample.

The FTIR analysis (FIG. 10b) of the PM₁₀ derived ND samples showing the presence of C—H, C—O, C═O, and O—H group predominantly which indicates that the PM₁₀ derived ND samples are highly functionalized and soluble in water.

Raman Analysis of ND samples from PM₁₀ (FIG. 10c) also shows similar results as of TSPM derived ND samples. This analysis shows the presence of both the characteristic bands of ‘G’ and ‘D’. Raman scattering peak at 1320-1440 cm⁻¹ indicates the presence of ND particle. This peak specifies the D band corresponding to the sp³ hybridized structure of carbon. The other peak observed at 1620 cm⁻¹ referred to as G-band attributed to the sp² structure of carbon. From this study, it is confirmed that the nanodiamonds fabricated from PM₁₀ contains both sp³ and sp² structure of carbon and dominance of diamond particles rather than the graphite.

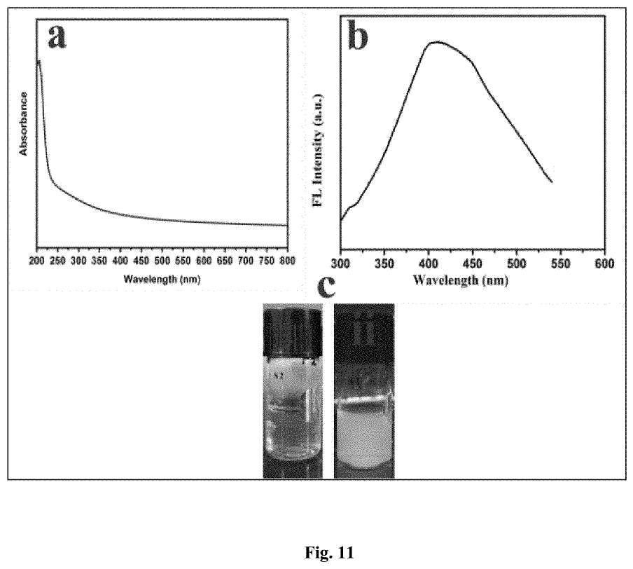

FIG. 11a shows the UV-visible spectroscopic analysis of the NDs samples obtained from PM₁₀ samples. The UV band was observed at 210 nm are due to the excitation of electrons from π-π* and n-π* transition of C═C and C═O bonds present in the NDs samples. The FL spectra (FIG. 11b) appeared at 420 nm, corresponding to blue fluorescence properties observed under UV-light at 365 nm (see FIG. 11c) as the similar results observed in TSPM derived ND sample.

### Example 3: Surface Properties of NDs Prepared from Air Pollutants (TSPM and PM₁₀)

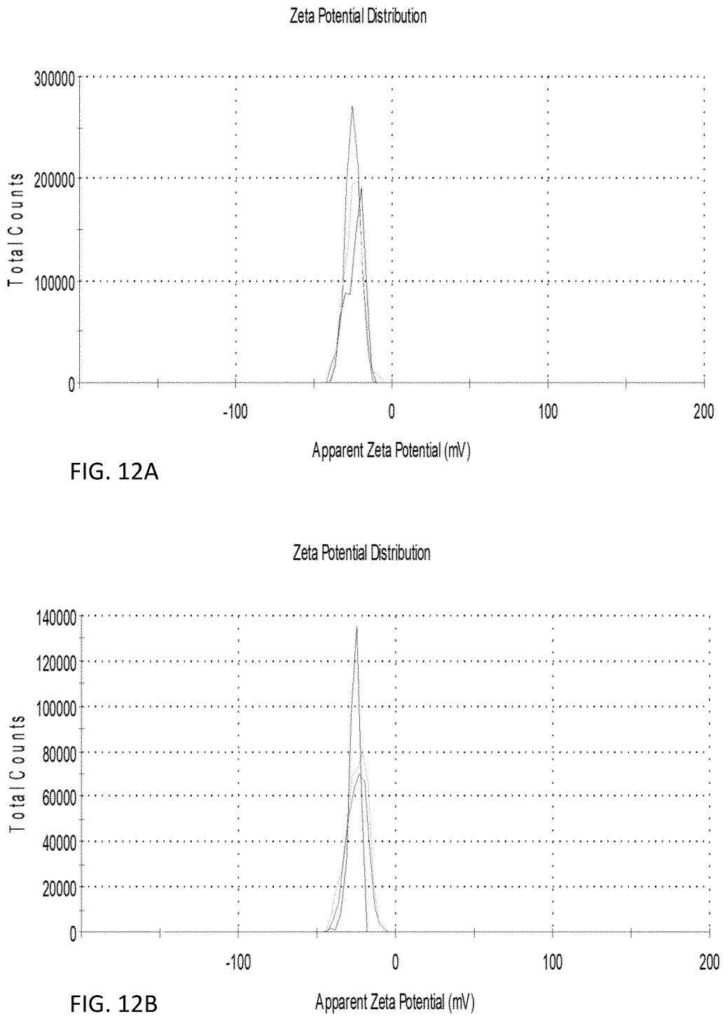

Zeta potential is the electrostatic potential or net charge at the plane of particle-slipping. Zeta potential analysis was carried out (ZETASIZER; Model-Nano ZS, Malvern, UK) to know the stability and surface charge of the PMs-derived NDs. The Zeta potentials of the NDs were found to be in the range of −24 mV to −25 mV (FIGS. 12a and 12b), which indicated that produced NDs suspensions are stable. The negative zeta potential values were observed due to the presence of oxygen-containing functional groups, such as carbonyl (C═O) and carboxyl (COOH) groups which are dissociated in the surface of diamond particles.

### Example 4: Toxicological Studies of NDs Prepared from Air Pollutants (TSPM and PM₁₀)

Toxicological Studies:

As the air pollutants are considered as the most dangerous to human health, hence the toxicity of derived NDs from these C-sources were evaluated to know whether the produced product are toxic or non-toxic. Cells were sustained in RPMI medium improved with 10% Fetal bovine serum (FBS) and antibiotics at 37° C. in culture flasks with 5% CO₂. Confluent monolayers (80%) of human normal kidney epithelial (NKE) cells were subjected to exposure of produced NDs at a dose of 5, 10, 20, 50, 100, 150, and 200 μg/mL for 24 hours.

Cytotoxicity Analysis and DNA Fragmentation or Genotoxicity Analysis:

Cytotoxicity was determined by using the Alamar Blue reduction bioassay. This method is based upon Alamar Blue dye reduction by live cells. After treatment with the produced NDs, the treatment medium was aspirated and 200 μL of Alamar Blue solution was added to each well and further incubated for 4 h at 37° C. The optical density of each well was measured by using a microplate reader with absorbance at 570 and 600 nm. Similar conditions were repeated three times and the well without any treatment was taken as a control. The results were expressed as a percentage over control.

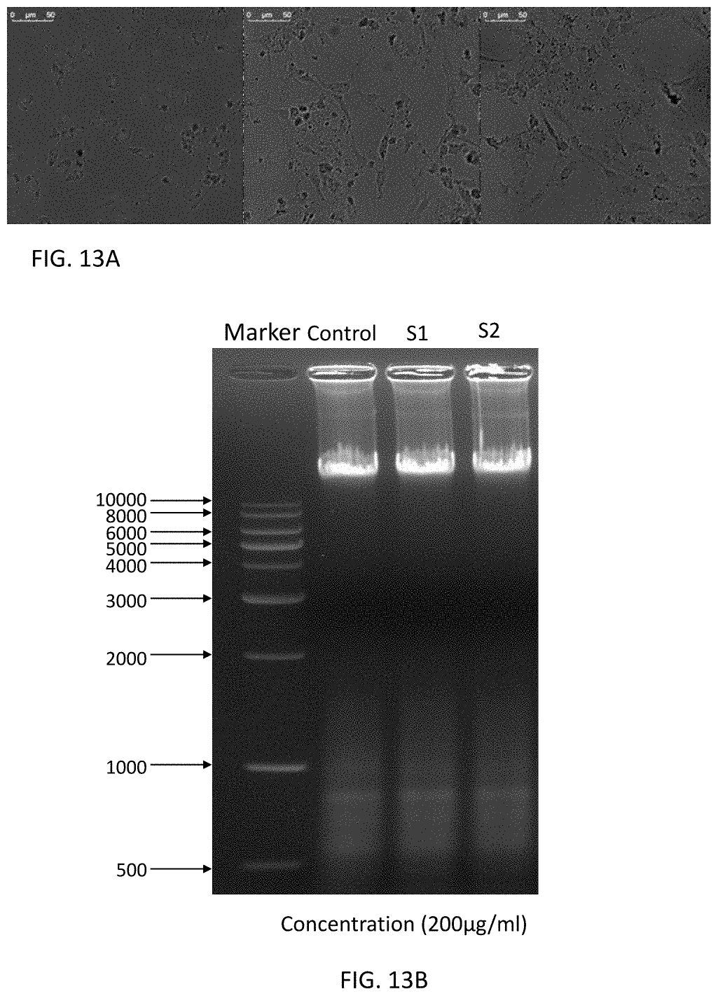

The cytotoxicity analysis was performed to evaluate the toxicity levels of air pollutants (TSPM and PM₁₀) derived NDs for their further utilization. The results demonstrated that NDs did not cause any change in the cell viability compared to those seen in the control study. FIG. 13a clearly shows that the cytotoxicity of NDs is considerably lower at all the concentration (i.e. 0-200 μg/mL). It revealed that the fabricated NDs are non-toxic on NKE cells. Thus, the air pollutants (TSPM and PM₁₀) could also be a source for the fabrication of non-toxic blue fluorescent NDs and process may lead to the mitigation of atmospheric particulate pollution.

The extent of genotoxicity or fragmentation of DNA was assayed in genomic DNA samples with the help of electrophoresis technique, isolated from control as well as produced NDs treated cells, on agarose/ethidium bromide gels. After treatment, cells were washed with PBS followed by fixation with paraformaldehyde and mounted with a coverslip using the mounting media containing DAPI. Images were observed by confocal microscopy with an inverted laser scanning confocal microscope (Leica Microsystems, Germany).

The DNA fragmentation pattern treated in different cells compounds were examined. It was observed that the treatment with different compounds at a dose of 200 μg/mL for 24 hours did not cause DNA fragmentation (see FIG. 13b).

In summary, it can be confirmed that the air pollutants derived nanodiamonds are non-toxic for human kidney cell-line and it also even non-toxic at genetic level. This process will lead to the mitigation of atmospheric particulate pollution.

The main advantages of the present invention are:
1. By using the process of the present invention, typical blue-fluorescent nanodiamonds can be easily produced as compared to other drastic and tedious physical/chemical methods such as hydrothermal synthesis, ion bombardment, laser bombardment, microwave plasma chemical vapor deposition techniques, ultrasound synthesis, and electrochemical synthesis.
2. Using the process of the present application, the toxic air pollutants (TSPM and PM₁₀) are converted into non-toxic high-value nanodiamonds (NDs).
3. Hydrogen peroxide used as oxidizing agent in the process is environment-friendly as compared to other chemical or acid solutions.
4. The process of the present invention is less time consuming.

1. A method for preparing nanodiamonds from atmospheric pollutants comprising the steps of:
a) collecting atmospheric pollutants;
b) mixing the atmospheric pollutants obtained in step (a) with hydrogen peroxide to obtain an oxidized particulate matter;
c) ultrasonicating the oxidized particulate matter obtained in step (b) to obtain a first mixture;
d) filtering the first mixture obtained in step (c) using a polytetrafluoroethylene membrane filter (0.22 μm) to obtain a filtrate;
e) centrifuging the filtrate obtained in step (d) to obtain a supernant;
f) treating the supernatant obtained in step (e) with HNO₃/H₂SO₄ acid and heating to obtain an extract;
g) neutralizing the extract obtained in step (f) by adding ammonia solution drop wise to obtain a second mixture;
h) filtering the second mixture obtained in step (g) using an ultrafiltration (1 KDa) to obtain a filtrate solution; and
i) concentrating the filtrate solution obtained in step (h) to obtain the nanodiamonds.

2. The method as claimed in claim 1, wherein the atmospheric pollutants comprise Total Suspended Particulate Matter having the particle size less than 100 μm (TSPM) and Particulate Matter having the size of aerodynamic diameter of 2.5-10 μm (PM₁₀).

3. The method as claimed in claim 1, wherein the atmospheric pollutants are collected using High Volume Sampler or Respirable Dust Sampler on quartz filter papers.

4. The method as claimed in claim 1, wherein the hydrogen peroxide used in step (b) is 30% (v/v).

5. The method as claimed in claim 1, wherein the ultrasonication in step (c) is carried out for 1 hour at room temperature.

6. The method as claimed in claim 1, wherein the centrifugation in step (e) is carried out at 1400 rpm for 1 hour.

7. The method as claimed in claim 1, wherein heating in step (f) is carried out at 55-60° C. for 30 min.

8. A nanodiamond having a diameter in the range of 3-24 nm and a X-ray diffraction pattern showing peaks at the d-spacings listed in Table A:
TABLE A

| ‘d’ spacing value (Å) | 2θ (°) |
|---|---|
| 2.0 | 43.22 |
| 1.26 | 75.1 |

9. The nanodiamond as claimed in claim 8 having oxygen-containing hydrophilic functional groups and blue-fluorescence under UV-light.

10. The nanodiamond as claimed in claim 8, wherein said nanodiamond are non-toxic and bio-compatible.

11. The nanodiamond as claimed in claim 8 for use in bio-sensing, biomedical imaging, drug delivery, wear resistant polymer, lubricant additives, and metal coating.
